(19) United States
(12) Patent Application Publication
(10) Pub. No.: US 2025/0341448 A1
(43) Pub. Date: Nov. 6, 2025

(54) MONITORING SYSTEM TO MONITOR AN ENVIRONMENT TO BE INVESTIGATED

(71) Applicants: REDOX S.R.L., Reggio Emilia (IT); MAKROS S.R.L., Ferrara (IT)

(72) Inventors: Angelo BONI, REGGIO EMILIA (IT); Cristian SALETTI, REGGIO EMILIA (IT); Matteo MONTANARI, SAN LAZZARO DI SAVENA (BO) (IT); Gian Luca PALADINI, CASINA (REGGION EMILIA) (IT); Massimo LUISE, FERRARA (IT); Federico BASSI, MONTECCHIO MAGGIORE (VICENZA) (IT)

(21) Appl. No.: 18/869,993

(22) PCT Filed: Jun. 7, 2023
(86) PCT No.: PCT/IB2023/055859

§ 371 Date: Nov. 27, 2024

(30) Foreign Application Priority Data
Jun. 7, 2022 (IT) 102022000012047

(51) Int. Cl. G01N 1/22; G01N 21/33; G01N 33/00

(52) U.S. Cl. CPC G01N 1/2273; G01N 21/33; G01N 33/0062

(57) ABSTRACT

A monitoring system (1) for monitoring an environment (A) to be investigated and a compactable containment system (100) equipped with the monitoring system (1); the monitoring system (1) comprises: at least one sensor (3) of environmental conditions for detecting at least the humidity and temperature of the environment (A) to be investigated; a verification assembly (4) to detect the presence of fungal cells in the environment (A) to be investigated; and a control unit (CU) configured to estimate a probability of fungal proliferation in the environment (A) to be investigated, as a function of what is detected by the detection assembly (2), and to activate the verification assembly (4) for values of the probability of fungal proliferation greater than or equal to a predefined limit value.

The present invention relates to the field of monitoring of conditions inside an environment for storing articles, such as for example books, journals, paintings, artworks of various types, etc.

More particularly, the present invention relates to the field of monitoring of environmental conditions inside compactable containment systems adapted to isolate the containment volume defined therein from the external environment and used, for example, inside libraries, museums, offices, . . . , for the storage of generally paper-based articles, to which the description that follows will make explicit reference, without for this reason losing its general character.

In this technical field, various compactable containment systems have been proposed over the years, such systems comprising a plurality of container modules arranged facing one another and reciprocally movable between a compact configuration, in which the container modules are in contact with one another so as to prevent access to the containment space and reduce the bulk of the compactable containment system to a minimum, and an access configuration, in which at least two container modules are spaced apart so that an access corridor is defined between the front openings of two successive container modules. To ensure the correct isolation of the containment space from the external environment, the walls of the various container modules are generally fitted with insulating panels and each container module has, along the perimeter edges of the respective front opening, isolating elements (for example groups of seals) adapted to ensure the isolation of the storage space from the external environment. Some known compactable containment systems of this type are described, for example, in patents ITFE20110003 and 102018000010669.

Such compactable containment systems, despite ensuring optimal performances in terms of isolating the internal containment space from the external environment, and thus of protecting the articles contained therein from external agents, such as dust, but also fire and/or water in the case of unforeseen events, and despite contributing significantly to improving the environmental conditions in the storage environment, for example in terms of humidity, have some limits.

In particular, such limits are connected to various situations (in terms of temperature and humidity) that can be created inside the various container modules with variations in the internal environmental conditions (which can change, for example, as a function of the type of insulating panels used, the closure seals, etc.) and/or the external environmental conditions (for example in terms of temperature, humidity, exposure to sunlight, etc.), but also due to the persistence over time of certain environmental conditions, for example caused by the absence or insufficient ventilation of the container modules, which can induce the so-called phenomenon of stagnation.

With variations in such situations, unfavourable environmental conditions may arise inside the storage space, which in themselves could induce (in the long term) the degradation of the stored articles or, worse, they could create an environment conducive to the proliferation of bacteria and fungi, with consequent damage to the stored articles.

To prevent such a risk, it is advisable to monitor the environmental conditions inside the containment volume of such compactable storage units and in general the area in which such storage units are placed. Such problems, it is understood, are of fundamental importance, especially when the compactable containment systems must contain particularly valuable or particularly delicate material; one need only think of paintings, paper-based material, etc., which under non-optimal conditions would risk deteriorating or being damaged, with huge economic and cultural damage. Accordingly, there exist some storage environment monitoring systems adapted to control the environmental conditions of an environment to be investigated, for example by monitoring humidity and temperature. Such monitoring systems are typically equipped with sensors adapted to detect temperature and humidity conditions and an electronic control system configured to provide warnings (for example to an operator) when certain limit values are reached in the environmental conditions. However, such monitoring systems of a known type have limits, among which we shall mention the following.

First of all, the monitoring systems of a known type are bulky and require a wired power supply and/or energy storage devices, such as batteries, located inside the storage environments, with all the connected drawbacks in terms of bulk, safety, etc.

Moreover, the monitoring systems of a known type have numerous performance limits. In fact, they are based on a punctual detection of humidity and temperature which does not take account of the changes in the environmental conditions over time.

In addition, the known monitoring systems are not capable of providing an estimate of the actual probability of biological proliferation, fungal proliferation in particular, much less of verifying the actual presence of cells, for example spores (reproductive cells) and/or bacteria and/or fungi, inside the environment to be monitored.

The monitoring systems of a known type can at most provide information about the punctual temperature and humidity conditions inside the containment system, on the basis of which it is possible to evaluate, automatically or manually, whether or not such conditions would be conducive to cell reproduction, for example to the proliferation of fungal cells.

However, it is evident that such estimates do not allow a real automatic monitoring of the risk of fungal proliferation, since they do not take account of the time of exposure to certain environmental conditions, nor are they able to verify and detect the actual presence of fungal cells and/or mould. Therefore, to date, the monitoring of storage environment conditions and the risk of fungal proliferation, as well as the adoption of actions aimed at lowering and/or eliminating that risk, are entrusted to operators who, once they know the parameters detected by the monitoring systems of a known type, manually perform calculations, visual inspections and/or manual measurements in order to assess the actual risk of fungal proliferation and the rate thereof in the case of proliferation already underway.

This, in addition to all the limitations in terms of precision, reliability, repeatability, etc., typical of manual operations, implies the need to plan out manual environmental monitoring operations, with a consequent increase in the time required and the management costs of the storage systems.

The object of the present invention is to provide a monitoring system for monitoring an environment to be investigated and a compactable containment system that enable the limits of the prior art to be overcome, at least in part.

In accordance with the present invention, there is provided a monitoring system for monitoring an environment to be investigated and a compactable containment system endowed with said monitoring system, according to what is claimed in the independent claim that follows and, preferably, in any one of the dependent claims depending directly or indirectly on the independent claims.

The claims describe preferred embodiments of the present invention, which form an integral part of the present description.

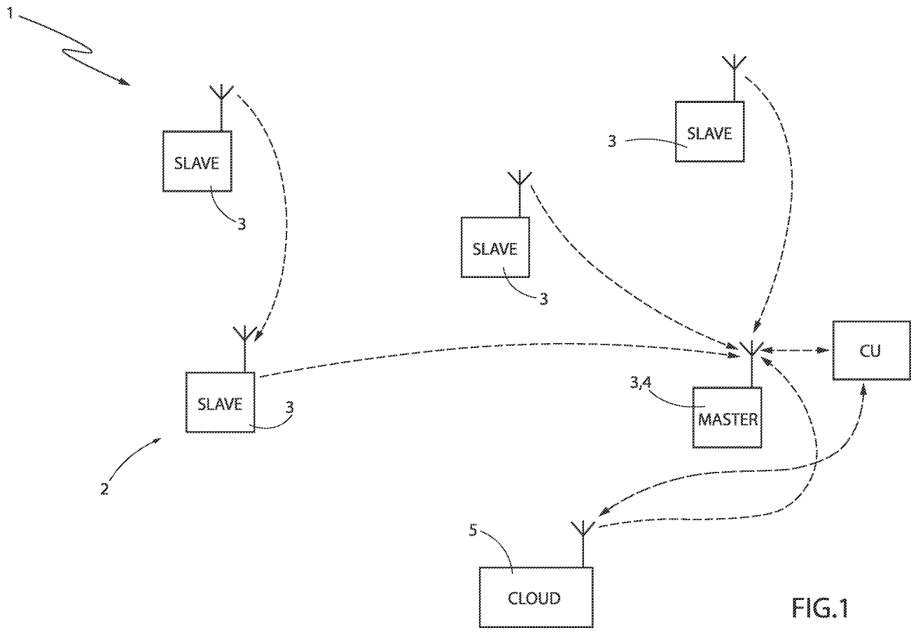

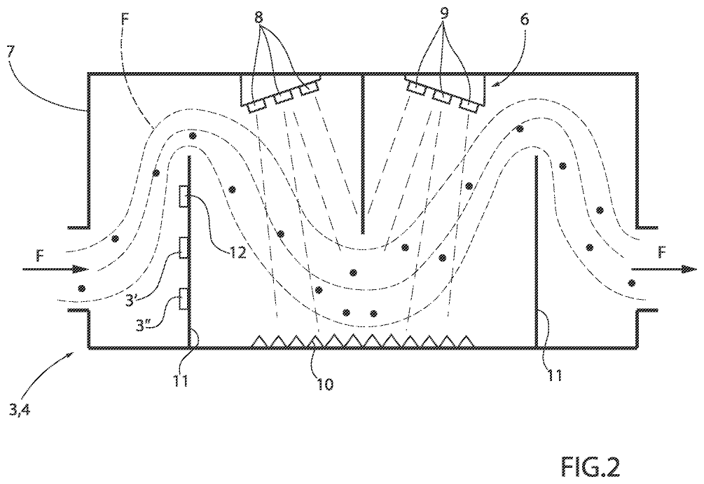

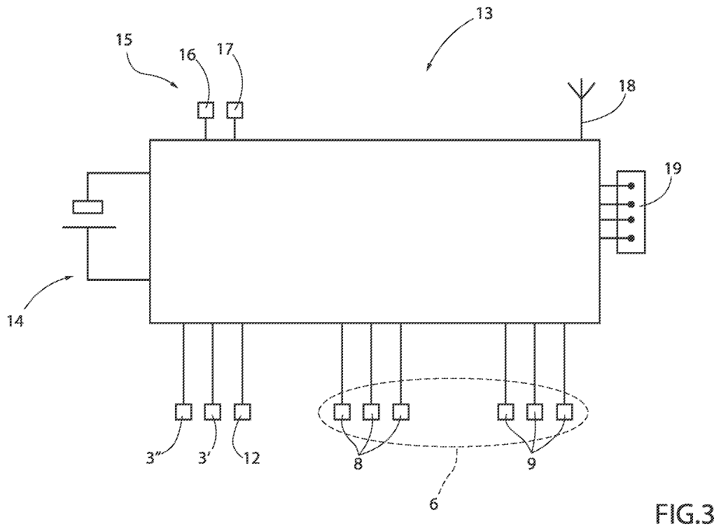

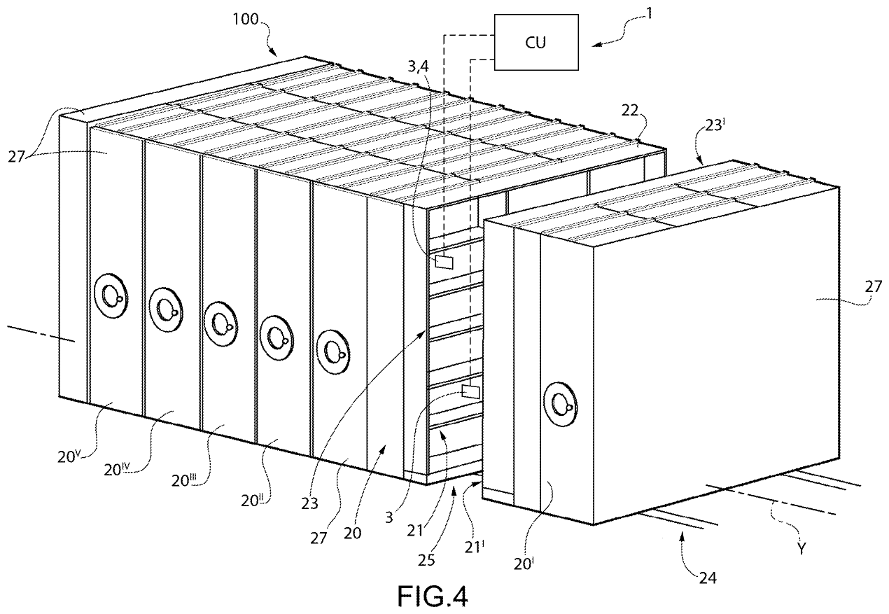

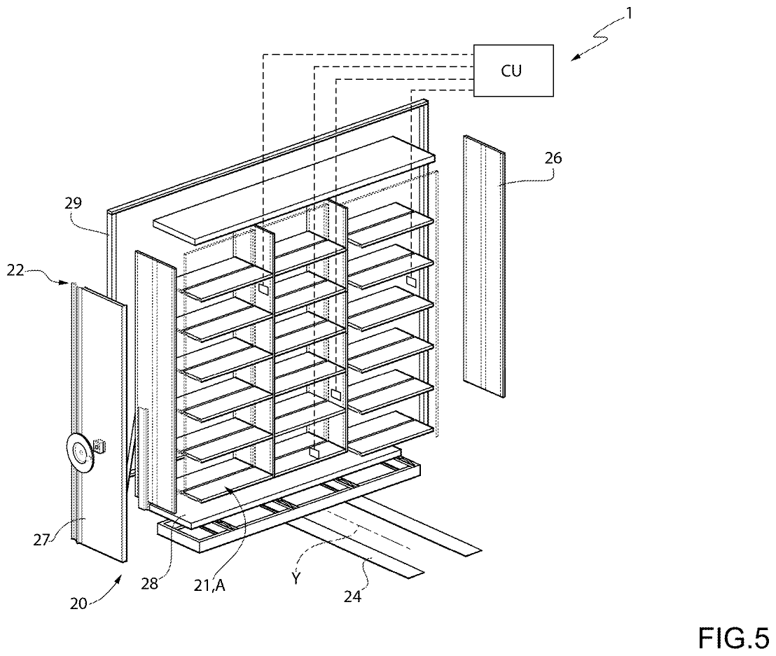

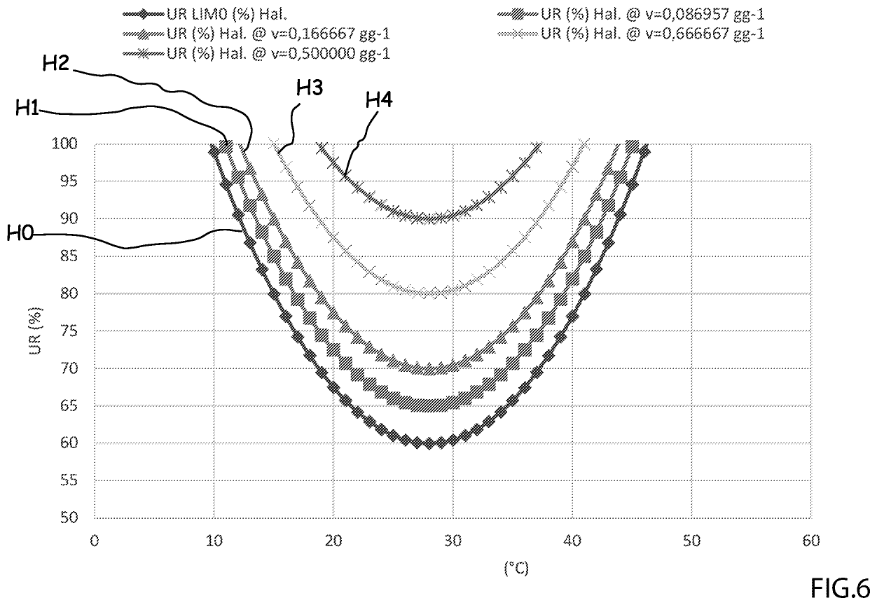

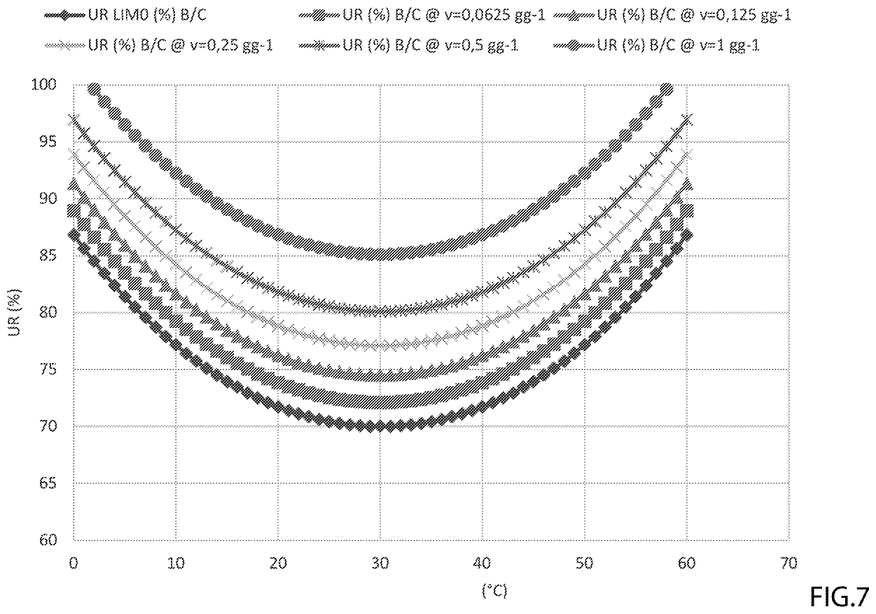

The invention is described below with reference to the appended drawings, which illustrate some non-limiting example embodiments, in which:
FIG. 1 illustrates a schematic representation of a monitoring system in accordance with one embodiment of the present invention;
FIG. 2 illustrates a schematic view of a sensor module that is part of the monitoring system in FIG. 1;
FIG. 3 illustrates a schematic view of a control processor of the sensor module in FIG. 2;
FIG. 4 is a perspective view of a compactable containment system in accordance with one embodiment of the present invention, with some details removed for the sake of clarity;
FIG. 5 is a partly exploded view of the compactable containment system in FIG. 4; and
FIGS. 6 and 7 are level curves or temperature and humidity isopleths which indicate the time necessary for the germination, respectively, of a fungus called Eurotium Halophilicum, typically present in storage environments and by now endemic throughout Europe, and of a class of fungi likewise spread throughout Europe, but prevalently in the north (group B/C), as a function of the temperature values (on the X-axis) and relative humidity (on the Y-axis).

In the appended figures, the number 1 denotes in its entirety a monitoring system for monitoring an environment A to be investigated. In particular (advantageously, but without limitation), the environment A to be investigated is an environment for storing articles, such as for example books, journals, paintings, artworks of varying kinds, etc., variously arranged inside the environment A to be investigated, for example placed in special compactable containment systems and/or in or on traditional furniture (e.g. shelves, display cases, etc.) and/or simply rested or hung in the environment A to be investigated. More in particular, according to some advantageous but not exclusive embodiments, the environment A to be investigated comprises (in particular is) the internal space of a compactable containment system 100 (FIG. 4), i.e. the (single) containment volume of the compactable containment system 100, as will be better explained below. Alternatively, or in combination, the environment A to be investigated comprises (in particular, is made up of) one or more rooms, in which articles are placed inside compactable containment systems and/or in or on traditional furniture as explained above.

The same reference numbers and letters in the figures identify the same elements or components with the same function.

Within the context of the present description, the term “second” component does not imply the presence of a “first” component. Such terms are in fact used as labels to improve clarity and should not be understood as limiting. The elements and features illustrated in the various preferred embodiments, including the drawings, can be combined together without going beyond the scope of protection of the present application as described below.

Advantageously, the monitoring system 1 comprises a detection assembly 2 comprising in turn at least one sensor 3 of environmental conditions configured to detect ambient parameters comprising at least the humidity and temperature of the environment A to be investigated. Preferably, the sensor 3 is configured to detect the relative humidity and temperature of the environment A.

The system 1 further comprises a verification assembly 4, configured to detect the presence of fungal cells (fungal spores in particular) in the environment A to be investigated.

The system likewise comprises a control unit CU, configured to define a probability of fungal proliferation in the environment A to be investigated, as a function of what is detected by the detection assembly 2, and to activate the verification assembly 4 if the probability of fungal proliferation is higher than a pre-established alert probability. In other words, the control unit CU is configured to activate the verification assembly 4 when the probability of fungal proliferation is greater than the pre-established alert probability. This makes it possible to verify the actual presence of fungal cells when the environmental conditions inside the environment A to be investigated are such as to warrant verification, allowing for an increase in the precision and reliability of the monitoring system 1 compared to the monitoring systems 1 of a known type, while at the same time limiting the electrical consumption of the verification assembly 4 and hence of the monitoring system 1. Physically, the control unit CU can be made up of a single device or a number of devices separate from one another and communicating via a wired or wireless network.

Preferably, but not necessarily, the probability of fungal proliferation is a function of a germination potential, understood as a portion of the germination process that develops in one day, for a given environmental condition of temperature and relative humidity. Preferably, but not necessarily, the probability of fungal proliferation substantially coincides with the germination potential.

The germination potential of numerous fungi is known in the literature. The germination potential of a fungus is obtained substantially from laboratory tests in which the spores are placed under pre-established environmental conditions of temperature and relative humidity. Such environmental conditions are maintained until spore germination occurs. The inverse of the time T it takes for spore germination, i.e. 1/T, indicates the germination potential of the fungus. In practical terms, assuming that the spores of a given fungus have germinated in three days, the germination potential of that fungus is equal to ⅓=0.33 per day, or 33% per day. By carrying out different tests under different environmental conditions of temperature and relative humidity, it is possible to construct, for a given target organism, a family of isopleth curves of the type represented in FIGS. 6 and 7.

In greater detail, and with reference to the graph in FIG. 6, each isopleth curve (H0,H1,H2,H3,H4) expresses the daily portion of germination of a spore of the target fungal organism Eurotium Halophilicum under a given environmental condition of temperature and relative humidity. The germination potential (V) can be expressed in terms of percentage per day: in practical terms, the curve H1 indicates a germination potential of about 8% per day. In other words, when remaining under the environmental conditions defined along the curve H1, the spore will germinate in about twelve days. In other words, every day in which the spore remains under an environmental condition defined along the curve H1 contributes 8% to the germination process.

Similarly, the curve H4 indicates a germination potential of about 66%, i.e. it indicates a set of temperature-relative humidity pairs in which a spore takes 1.5 days to germinate. Under the environmental conditions according to the curve H4, every day contributes 66% to the germination process. The germination potential is thus much higher compared to the curve H1.

The curve H0 substantially indicates a lower limit, below which spores cannot germinate. For all the temperature-relative humidity pairs below the curve H0 we have no increase in the germination potential.

Each point inside the area of the isopleth whose lower limit is represented by the curve H0 contributes to increasing the germination potential until the total germination time (potential≥1) that defines the alert probability is reached.

Preferably, ma not necessarily, the alert germination potential is calculated on the basis of the family of isopleths of the target fungus Eurotium Halophilicum (FIG. 6), typically present in indoor environments in Mediterranean and central-southern European regions and apt to proliferate at low levels of humidity and temperature values comprised between about 15° and 30° C., the optimum value being 28° C., or the family of isopleths of the B/C group of fungi widely present in indoor environments in colder regions typical of northern Europe and belonging to the classes indicated in FIGS. 7 as B (pathogenic under long exposure) and C (pathogens that are not harmful to humans but cause damage to heritage), apt to proliferate at higher values of humidity compared to the fungus Eurotium Halophilicum, but with more abrupt germination potentials. By means of a user interface (not illustrated) included in the monitoring system 1, it is possible to choose on which family of curves to calculate the alert germination potential, for example based on the geographic position and/or type of environment A to be investigated and/or type of articles contained in the environment A to be investigated and/or the level of safety it is intended to maintain, etc. According to some advantageous but non-limiting embodiments, the detection assembly 2 is configured to detect the humidity (in particular, relative humidity) and temperature of the environment A to be investigated at pre-set time intervals (1 hour) and the control unit CU is configured to calculate, at every pre-set time interval, the value of the abovementioned germination potential: for every temperature-relative humidity pair falling within the area of the families of isopleths considered, the hourly portion of the germination process is calculated mathematically and added to the value of the potential reached at the previous hourly reading.

In order to enable the activity of comparison performed by the control unit CU, it is thus not necessary to know exactly the germination potential corresponding to all the pairs of ambient temperature and humidity parameters falling within the area of the isopleths, but it is rather sufficient to have a limited family of isopleth curves from which it is possible to interpolate the increase in potential.

In even greater detail, advantageously, but without limitation, the control unit CU is configured to reduce the abovementioned germination potential by a given percentage after a certain period of time in which the value thereof has remained constant.

According to some advantageous but non-limiting embodiments, the control unit CU is configured to apply a first corrective coefficient when the value of the germination potential remains constant for a first period of time, and a second corrective coefficient when the value of the potential remains constant for a second period of time, longer than the first period of time. Advantageously, but without limitation, the first corrective coefficient is greater than the second corrective coefficient.

In some preferred non-limiting cases, the control unit CU is configured to apply to the germination potential a corrective coefficient equal to X.0.66 (where X=value of the potential reached) when the calculated value of the potential remains constant (does not increase) for a period longer than 30 days and up to 60 days, and a corrective coefficient equal to X.0.33 when the value of the germination potential remains constant (no increase) for a period of 60 days and up to 90 days. After 90 days of constant potential (no increase), the calculated value of the germination potential becomes zero. According to some advantageous but non-limiting embodiments, as schematically illustrated in FIGS. 1, 4 and 5, the detection assembly 2 comprises a plurality of sensors 3 of environmental conditions. In detail (advantageously, but without limitation), each of said sensors 3 of environmental conditions in turn comprises at least one temperature detector 3′ and one humidity detector 3″, advantageously a relative humidity detector 3″ (see for example FIG. 2).

Preferably, ma not necessarily, each of said sensors 3 of environmental conditions of the detection assembly 2 is configured to exchange data with the other sensors 3 of environmental conditions and with the control unit CU in a wireless (or alternatively a partially or totally wired) mode. Even more advantageously, but without limitation, the various sensors 3 of environmental conditions each comprise a communication module for exchanging data via radio or via Bluetooth both with the other sensors 3 of environmental conditions, and with the control unit CU (see FIG. 1). Furthermore, advantageously but without limitation, the monitoring system 1 comprises a data collection system, for example a memory 5, according to some cloud-based embodiments, containing at least the abovementioned level curves or isopleths of the biological risk, and configured to store the data collected by the detection assembly 2 and the data collected by the verification assembly 4.

According to some advantageous but non-limiting embodiments, the verification assembly 4 is configured to have a flow F of air to be analysed (schematically illustrated in FIG. 2) pass through it and comprises at least one analysis unit 6 configured to analyse the flow F of air and to detect the presence of fungal cells in that flow F of air.

Preferably, but not necessarily, as illustrated in FIG. 2, the verification assembly 4 comprises a casing 7 configured to have a flow F of air to be analysed pass through it.

According to some non-limiting embodiments, like the one illustrated in FIG. 2, the casing 7 is also configured to shield against external light. In particular, the verification assembly 4 (and the analysis unit 6) comprises at least one light emitter 8 configured to emit a beam of ultraviolet light (schematically illustrated in FIG. 2) having a pre-set wavelength, for example from about 200 nm to about 400 nm (optionally also variable), in particular from about 250 nm to about 350 nm, towards the flow F of air to be analysed, and at least one photodetector 9 configured to detect the presence of fungal cells in the flow F of air, in particular (advantageously but without limitation) based on fluorescence and/or direct optical reflection. According to other unillustrated, non-limiting embodiments, the light emitter 8 is configured to emit a beam of light other than ultraviolet light, for example visible, infrared, microwave or radio.

According to further non-limiting embodiments, inside the casing 7, the flow F of air is analysed by means of an electronic nose and/or a mass spectrometer. According to still other embodiments, the flow F of air is analysed on the basis of the absorption of the light beam passing through the flow F of air.

It remains understood that the analysis unit 6 could be constructed in any other way that allows the identification of fungal cells (spores in particular). According to some advantageous but non-limiting embodiments, the casing 7 of the verification assembly 4 is made of opaque, non-reflective black material. Even more advantageously, but without limitation, the casing 7 has at least one corrugated wall 10 arranged and configured so as to define a further barrier to reflected light. Alternatively, or in combination (according to some advantageous but non-limiting embodiments), the verification assembly 4 (in particular, the casing 7 of the verification assembly 4) comprises a plurality of partitions 11 arranged and configured so as to induce the flow F of air to follow a given path in order to improve the efficiency of the analysis unit 6 (see FIG. 2).

Advantageously but without limitation, the verification assembly 4 further comprises at least one gas detector 12 to detect the aforesaid flow F of air and activate the abovementioned analysis unit 6.

According to some advantageous but non-limiting embodiments, the detection assembly 2 (even more particularly, at least one of the sensors 3 of environmental conditions) comprises the verification assembly 4. In other words, according to some advantageous but non-limiting embodiments, the sensor 3 of environmental conditions and the verification assembly 4 are made in a single body.

According to the non-limiting embodiment in FIG. 3, the verification assembly 4 comprises at least one control processor 13 configured to manage the analysis unit 6 (in particular the various light sources 8, and the photodetectors 9), the temperature and humidity detectors 3′, 3″ and the optional gas detector 12.

Advantageously, but without limitation, the control processor 13 further comprises (or is connected to) a battery 14, advantageously of the rechargeable type, to power all the functions of the verification assembly 4 and, advantageously, but without limitation, at least one status signalling unit 15 configured to signal when the verification assembly 4 is activated. It remains understood that according to other advantageous but non-limiting embodiments, the verification assembly 4 could also comprise a disposable battery, or in any case a battery of the non-rechargeable type.

In the advantageous but non-limiting embodiment illustrated in FIG. 3, the control processor 13, in particular the status signalling unit 15, comprises two LEDs 16 and 17, each selectively activatable based on the status of the verification assembly 4. In detail, when the verification assembly 4 is comprised in the environmental sensor 3 (i.e. when the verification assembly 4 is made in a single body with the environmental sensor 3) the control processor 13 is configured to selectively activate the LEDs 16 and 17 based on the operation of the single module made up of the verification assembly 4 and the environmental sensor 3 (for example by switching on the LED 16 when only the temperature and humidity detectors 3′, 3″ are active, and the LED 17 when the analysis unit 6 and optionally the gas detector 10 are also active).

Advantageously, but without limitation, the control processor 13 also comprises an antenna 18 to enable communication with the other environmental sensors 3 and/or with the control unit CU and a connector 19 configured to allow a wired connection with the other environmental sensors 3 and/or to allow charging of the battery 14.

According to some advantageous but non-limiting embodiments, the monitoring system 1 further comprises a warning device (not illustrated) that may be activated by the control unit CU based on what is detected by the detection assembly 2 and/or what is detected by the verification assembly 3. For example, the control unit CU is configured to activate the warning device so as to emit an alarm signal when the predefined limit value of the probability of fungal proliferation is reached and/or when the verification assembly 4 detects the presence of fungal cells in the environment A to be investigated. Alternatively, the control unit CU is configured to activate the warning device so as to emit a first alarm signal when the predefined limit value of the probability of fungal proliferation is reached and/or a second alarm signal, different from the first alarm signal, when the verification assembly 4 detects the presence of fungal cells in the environment A to be investigated.

According to yet other advantageous but unillustrated, non-limiting embodiments, the monitoring system 1 further comprises an environmental regulator assembly which is configured to vary the environmental conditions of the environment A to be investigated and comprises at least one of a humidifier device, a conditioning device, a forced ventilation device, and an air purifier device, advantageously connected to the control unit CU. In this case, advantageously, but without limitation, the control unit CU is configured to command the activation of the environmental regulator assembly for a given time determined as a function of what is detected by the detection assembly 2 and/or what is detected by the verification assembly 3 so as to vary the conditions inside the environment A to be investigated. According to some advantageous but non-limiting embodiments, said environmental regulator assembly is placed inside the environment A to be investigated. In addition, or alternatively, the environmental regulator assembly is placed outside the environment A to be investigated in an area that is selectively connectable with the environment A to be investigated, for example in an adjacent space that can be placed in fluid communication with the environment A to be investigated. The presence of the environmental regulator assembly makes it possible to maintain, inside the environment A to be investigated, a value of the probability of fungal proliferation lower than the limit value.

In accordance with a second aspect of the present invention, a compactable containment system 100 equipped with the above-described monitoring system 1 is presented.

With particular reference to FIGS. 4 and 5, the compactable containment system 100 comprises: a first container module 20 slidable along a sliding direction Y, and which comprises, in turn, a respective containment compartment 21, a respective outer casing 22 which delimits (laterally, at least partially) the containment compartment 21, and a respective access opening 23 to allow access to the containment compartment 21; and at least a second container module 20I that is arranged facing the first container module 20, is slidable along the sliding direction Y in order to be moved closer to or farther away from the container module 20, and comprises, in turn, a respective containment compartment 21I, a respective outer casing 22I which delimits (laterally, at least partially) the containment compartment 21I, and a respective access opening 23I which is facing the access opening 23 and is configured to allow access to the containment compartment 21I. Advantageously, but without limitation, the compactable containment system 100 comprises a plurality of container modules 20, 20I, 20II, 20III, 20IV and 20V which are reciprocally movable along the advancement direction Y (see FIG. 4). More advantageously, with particular reference to FIG. 4, the container modules 20, 20I, 20II, 20III, 20IV and 20V are arranged facing one another in line along the sliding direction Y and can be activated to move closer to or farther away from one another.

Advantageously, the containment system 100 further comprises a movement assembly 24 that may be activated so as to allow the reciprocal movement of the container modules 20, 20I, 20II, 20III, 20IV and 20V between an open configuration (see the reciprocal position of the container modules 20 and 20I in FIG. 4), in which the successive container modules 20 and 20I are arranged at a reciprocal distance along the sliding direction Y, such that between the first access opening 23 and the second access opening 23I an access corridor 25 is defined, and a compact configuration (see the reciprocal position of the container modules 20, 20I, 20II, 20III, 20IV and 20V in FIG. 4), in which the successive container modules 20, 20I, 20II, 20III, 20IV and 20V are arranged in contact with one another at the respective access openings 23, 23I so as to delimit a single containment volume, and advantageously isolate it from the external environment.

In detail, advantageously, but without limitation, in the compact configuration, the successive container modules 20, 20I, 20II, 20III, 20IV and 20V abut against one another (in particular at the perimeter of the respective access openings 23, 23′) so that each container module 20, 20I, 20II, 20III, 20IV and 20V delimits (and closes off) the access opening 23, 23I of the adjacent container module 20, 20I, 20II, 20III, 20IV and 20V, thereby isolating the abovementioned single containment volume from the external environment.

To this end, advantageously but without limitation, the abovementioned outer casings 22, 22I of each container module 20, 20I, 20II, 20III, 20IV and 20V each comprise at least one respective insulating layer 26 (in particular fire resistant), advantageously arranged so as to delimit the containment compartment 21, 21I of the respective container module 20, and 20I with respect to the external environment (see FIG. 5). Advantageously, but without limitation, the insulating layer 26 comprises (in particular, is made of of) an insulating panel made of a material comprising calcium silicate and/or metal materials and/or glass wool and/or rock wool and/or wood wool, optionally coated, painted with fire-resistant and/or flame retardant paints, and/or optionally with fire-resistant and/or flame retardant substances added to them.

In even greater detail, according to some advantageous but non-limiting embodiments, the outer casings 22, 22I of each container module 20, 20I, 20II, 20III, 20IV and 20V each comprise a respective outer structure 27, and a respective inner structure 28 which are arranged spaced apart so that between them a space for receiving the insulating layer 26 is defined and are configured (in particular, dimensioned and shaped) to be joined together to define a coupling of mating parts. In even greater detail, they are configured (in particular, dimensioned and shaped) to form an interstitial labyrinth seal at a perimeter edge of each container module 20, 20I, 20II, 20III, 20IV and 20V.

According to some embodiments, the compactable containment system 100 further comprises at least one isolation assembly 29 arranged and configured so as to sealingly isolate the abovementioned (single) containment volume from the external environment in the compact configuration.

Advantageously, but without limitation, the isolation assembly 29 is arranged at a perimeter edge of the container modules to ensure a sealing isolation of the containment volume from the external environment in the compact configuration (see for example FIG. 5). Even more advantageously, but without limitation, the isolation assembly 29 comprises at least one seal comprising (in particular made of) a material selected from an intumescent and/or fire-resistant and/or flame-retardant material.

It remains understood that the isolation assembly 29 could comprise any other element and combination of elements suitable for ensuring the sealed isolation of the (single) containment volume from the external environment. According to some advantageous but non-limiting embodiments, the compactable containment system 100 is made in accordance with what is described in any of the patents ITFE20110003 and 102018000010669, EP2497389, PCT/IB2019/060280, EP3886647 and EP3361906.

As mentioned above, advantageously, the compactable containment system 100 further comprises the monitoring system 1 to determine a possible fungal proliferation inside the containment volume thereof and a processing unit (not visible in the appended figures and known per se) which is in communication with the monitoring system 1 in order to receive information about the environmental conditions at least of the containment volume and is configured to command the activation of the movement assembly 24 towards the open configuration (or towards the compact configuration) for a time interval determined as a function of the information received by the monitoring system 1.

According to some advantageous but non-limiting embodiments, such as the ones illustrated in FIGS. 4 and 5, the processing unit of the compactable containment system 100 comprises (in particular, coincides with) the control unit CU.

In detail, advantageously but without limitation, the monitoring system 1 is arranged so as to monitor the environmental conditions both inside the compactable containment system 100 and outside, so as to assess the possible risk of fungal proliferation and command, where appropriate, the activation of the movement assembly 24. In other words, the monitoring system detects the environmental conditions also outside the abovementioned (single) containment volume so as to command the passage towards the open configuration only when the environmental conditions outside that (single) containment volume are better compared to the outside.

Advantageously, the monitoring system 1 is made in accordance with any one of the embodiments disclosed above, that is, it comprises a detection assembly 2, made according to any one of the embodiments disclosed above, in order to detect at the least humidity and temperature of the environment A to be investigated; a verification assembly 4, made according to any one of the embodiments disclosed above, in order to detect the presence of fungal cells in each containment compartment 21, 21I; and a control unit CU configured to estimate a probability of fungal proliferation as a function of what is detected by the detection assembly 2, and to activate the verification assembly 4 for values of the probability of fungal proliferation that are greater than or equal to a predefined limit value.

In particular, in this case (advantageously, but without limitation) the verification assembly 4 of the monitoring system 1 is arranged in the environment A to be investigated in a position such as to be passed through by the flow F of air that exits from the compactable containment system 100. According to some advantageous but non-limiting embodiments, the monitoring system 1 comprises at least one sensor 3 for detecting the environmental conditions for every containment compartment 21, 21I and configured to detect, at pre-set time intervals, at least the temperature of the respective containment compartment 21, 21I; and the processing unit is configured to command the activation of the movement assembly 24 in such a way as to induce the passage to the compact configuration, when the temperature detected and/or the humidity detected in at least one of the containment compartments 21, 21I exceeds, respectively, a threshold temperature value and/or a threshold humidity value. This ensures protection of the articles contained in the containment system 100, in the event of fire.

According to some advantageous but non-limiting embodiments, the verification assembly 4 of the monitoring system 1 is arranged at the base of each containment compartment 21, 21I.

According to some advantageous but non-limiting embodiments, the compactable containment system 100 comprises a signalling system (not illustrated) in communication with the processing unit and configured to provide an alarm signal, when the temperature inside at least one container module 20, 20I reaches the threshold temperature value and/or when the warning device of the monitoring system 1, if provided, is activated to emit a warning signal.

Advantageously, but without limitation, the processing unit is configured to compare, at every pre-set time interval, a last detected temperature and humidity value with the current temperature and humidity; and activate the abovementioned warning device to emit a first warning signal (alert) in case of temperatures and/or humidity exceeding the threshold temperature value and/or threshold humidity value and/or temperature variations greater than a given value, for example equal to (or greater than) about 2° C.

Advantageously, but without limitation, each containment module 20, 20I comprises a respective ventilation device that can be selectively activated to allow or prevent a flow of air from the external environment towards the containment compartment 21, 21I, and vice versa; and the processing unit is configured to selectively activate the ventilation device based on the information received from the monitoring system 1.

Alternatively, or in addition, according to some advantageous but unillustrated, non-limiting embodiments, each containment module 20, 20I comprises a further environmental regulator assembly (advantageously of the type described above with reference al monitoring system 1) comprising at least one of a humidifier device, a conditioning device, a forced ventilation device, and an air purifier device; the processing unit being configured to command the activation of the environmental regulator assembly for a time determined as a function of the information received from the monitoring system 1.

The presence and automatic activation of the ventilation device and/or of the environmental regulator assembly makes it possible to act automatically to maintain, inside each containment compartment 21, 21I, or more in general inside the environment A to be investigated (as also explained above), a value of the probability of fungal proliferation that is lower than the limit value (also based on the material contained in each containment compartment 21, 21I).

It remains understood that, according to some advantageous but non-limiting embodiments, the monitoring system 1 could also comprise detectors of other parameters, such as for example the moisture content of the articles and/or the presence of specific volatile compounds etc. and the control unit CU can be configured to estimate the value of the probability of fungal proliferation also as a function of said further parameters and optionally of the concentration of fungal spores.

Although the above-described invention makes particular reference to a very precise example embodiment, it should not be considered limited to that example embodiment, as all variants, modifications or simplifications covered by the appended claims, such as for example a different configuration of the compactable storage system, a different regulator assembly, a different type of sensors 3, etc., fall within the scope thereof. The monitoring system 1 and the compactable storage system 100 of the present invention have numerous advantages, among which we shall mention the following.

The monitoring system 1 and the storage system 100 comprising it enable precise, reliable monitoring of the environmental conditions in the environment A to be investigated, in particular in the single containment volume delimited by the compactable containment system 100, thereby assuring the correct conservation of the articles contained therein and preventing the risk of proliferation of fungal cells which could damage such articles.

1. A monitoring system (1) for monitoring an environment (A) to be investigated;
the monitoring system (1) comprises:
a detection assembly (2) in turn comprising at least one sensor (3) of environmental conditions configured to detect at least the humidity and temperature of said environment (A) to be investigated;
a verification assembly (4) configured to detect the presence of fungal cells in said environment (A) to be investigated;
a control unit (CU) configured to estimate a probability of fungal proliferation in said environment (A) to be investigated as a function of what is detected by said detection assembly (2) and to activate said verification assembly (4) for values of the probability of fungal proliferation greater than or equal to a predefined limit value.

2. The monitoring system (1) according to claim 1, wherein said control unit (CU) is configured to estimate said probability of fungal proliferation by comparing what is detected by the detection assembly (2) with one or more isopleth curves of germination potential, each of which associates a portion of the germination process, which develops in one day, of at least one target fungus, with pairs of temperature and humidity values.

3. The monitoring system (1) according to claim 1, wherein:
said detection assembly (2) is configured to detect the humidity and temperature of said environment (A) to be investigated at pre-set time intervals; and
said control unit (CU) is configured to detect the variation in the germination potential in a period of time, and to determine a corrective coefficient to be applied to the germination potential; said corrective coefficient being determined as a function of the detected variation in the germination potential.

4. The monitoring system (1) according to claim 3, wherein said control unit (CU) is configured to:
compare, at every pre-set time interval, a last value of the germination potential detected with the value of the germination potential detected at the preceding time interval; and
reduce the germination coefficient by a given percentage after a certain period of time in which said germination potential remains constant.

5. The monitoring system (1) according to claim 4, wherein said control unit (CU) is configured to reduce the germination coefficient by applying a first corrective coefficient when said detected germination potential remains constant for a first period of time, and a second corrective coefficient, when said value of the germination potential remains constant for a second period of time; the first corrective coefficient being greater than the second corrective coefficient and the first period of time being shorter than the second period of time.

6. The monitoring system (1) according to claim 1, wherein said verification assembly (4) is configured to have a flow (F) of air to be analysed pass through it and comprises at least one analysis unit (6) configured to analyse said flow (F) of air and detect the presence of fungal cells in said flow (F) of air.

7. The monitoring system (1) according to claim 6, wherein said verification assembly (4) comprises: a casing (7) configured to have the flow (F) of air to be analysed pass through it and to shield against external light, at least one light emitter (8) configured to emit a beam of ultraviolet light having a pre-set wavelength towards said flow (F) of air to be analysed, and at least one photodetector (9) configured to detect the presence of fungal cells in said flow (F) of air.

8. The monitoring system (1) according to claim 1, wherein said detection assembly (2) comprises said verification assembly (4).

9. The monitoring system (1) according to claims claim 1, comprising at least one warning device; said control unit (CU) being configured to command said warning device based on what is detected by said detection (2) and/or what is detected by said verification assembly (3).

10. The monitoring system (1) according to claim 1, comprising an environmental regulator assembly that is configured to vary the environmental conditions of the environment (A) to be investigated and comprising at least one of a humidifier device, a conditioning device, a forced ventilation device, and an air purifier device; said control unit (CU) being configured to command the activation of the environmental regulator assembly for a given time based on what is detected by said detection assembly (2) and/or what is detected by said verification assembly (3).

11. A compactable containment system (100) comprising:
a first container module (20) which is slidable along a sliding direction (Y), and comprises a first containment compartment (21), a first outer casing (22) which delimits at least partially the first containment compartment (21), and a first access opening (23) to allow access to the first containment compartment (21);
at least a second container module (20I) that is arranged facing the first container module (20), is slidable along the sliding direction (Y), and comprises, in turn, a second containment compartment (21I), a second outer casing (22I), which delimits the second containment compartment (21I), and a second access opening (23I), which is facing the first access opening (23) and is configured to allow access to the second containment compartment (21I);
a movement assembly (24) that may be activated so as to allow the reciprocal movement of said first container module (20) and second container module (20I) between an open configuration, in which said first container module (20) and second container module (20I) are arranged at a reciprocal distance along the sliding direction (Y), such that between the first access opening (23) and the second access opening (23I) an access corridor (25) is defined, and a compact configuration, in which said first container module (20) and second container module (20I) are arranged so as to delimit a single containment volume;
a monitoring system (1), made according to claim 1, for determining a possible fungal proliferation inside said single containment volume; and
a processing unit that is in communication with said monitoring system (1) and is configured to command the activation of said movement assembly (24) towards the open configuration for a time interval determined as a function of the information received by the monitoring system (1).

12. The compactable containment system (100) according to claim 11, wherein the monitoring system (1) comprises at least one sensor (3) for detecting the environmental conditions for every containment compartment (21, 21I) and configured to detect, at pre-set time intervals, at least the temperature of the respective containment compartment (21, 21I); and the processing unit is configured to command the activation of the movement assembly (24) so as to induce the passage to the compact configuration when the temperature detected and/or the humidity detected in at least one of the containment compartments (21, 21I) exceeds, respectively, a threshold temperature value and/or a threshold humidity value.

13. The compactable containment system (100) according to claim 11, wherein: each containment module (20, 20I) comprises a respective ventilation device which can be selectively activated so as to allow or prevent a flow of air from the external environment towards the containment compartment (21, 21I), and vice versa; and the processing unit is configured to selectively activate said ventilation device based on the information received by the monitoring system.

14. The compactable containment system (100) according to claim 11, comprising a second environmental regulator assembly comprising at least one of a humidifier device, a conditioning device, a forced ventilation device, and an air purifier device; the processing unit being configured to command the activation of the second environmental regulator assembly for a given time based on the information received by the monitoring system.

15. The compactable containment system (100) according to claims 11, wherein:
the first outer casing (22) and the second outer casing (22I) comprise at least one respective insulating layer (26);
said compactable containment system (100) comprises at least one isolation assembly (29) arranged and configured so as to sealingly isolate said single containment volume from the external environment, in the compact configuration.

16. The compactable containment system (100) according to claim 15, wherein said isolation assembly (29) comprises at least one seal comprising an intumescent material and/or a fire-resistant material and/or a flame-retardant material.

17. The compactable containment system (100) according to claim 15, wherein:
the first outer casing (22) and the second outer (22I) casing each comprise a respective outer structure (27), and a respective inner structure (28) which are arranged spaced apart so that between them a space for receiving the insulating layer (26) is defined and are configured to be joined together to define a coupling of mating parts.
